(19) United States
(12) Patent Application Publication
(10) Pub. No.: US 2009/0234265 A1
(43) Pub. Date: Sep. 17, 2009

(54) Compression Adjustable Fabric and Garments

(76) Inventors: Lawrence G. Reid, JR., Germanton, NC (US); Edward Grant, Raleigh, NC (US); Meghan Sarah Hegarty, Raleigh, NC (US); Frederick Livingston, Raleigh, NC (US)

Correspondence Address: KILPATRICK STOCKTON LLP, 1001 WEST FOURTH STREET, WINSTON-SALEM, NC, 27101, US

(21) Appl. No.: 12/402,646

(22) Filed: Mar. 12, 2009

Related U.S. Application Data
(60) Provisional application No. 61/036,099, filed on Mar. 13, 2008.

(51) Int. Cl. A61F 13/00; A61B 5/02; A61B 5/00; D04B 1/24

(52) U.S. Cl. 602/61; 600/504; 600/301; 602/76; 66/171

(57) ABSTRACT

A compression adjustable fabric and/or garment, system, and/or method can include a compression element (a) integrated into a fabric structure, (b) having a compressive pressure capability independent from compressive pressure capabilities inherent in the fabric structure, and (c) adjustable to provide various compressive pressures. In such a garment, the compressive pressure provided by the garment can be adjusted in all or part of the garment while the garment is being worn. The compressive pressures in different parts of the garment may be independently adjustable. The compression element can further comprise an inflatable tube and/or an electrically stimulatable yarn. The compression element can be integrated into the fabric structure by being knit into, or laid in, the fabric structure.

### CROSS-REFERENCE TO RELATED APPLICATION

This application claims benefit of U.S. Provisional Patent App. No. 61/036,099, filed Mar. 13, 2008, which application is incorporated by reference herein in its entirety.

### FIELD OF THE INVENTION

The present invention relates to a compression adjustable fabric and garments made from compression adjustable fabric. Such compression adjustable fabric and garments may be useful for dynamically adjusting compressive pressure at different locations on a person's body while the fabric or garment is being worn.

### BACKGROUND OF THE INVENTION

In some conventional compression garments, the compressive force that the garment is capable of generating can be provided by various yarn and construction factors. Such factors can include, for example, yarn type and size, characteristics of stretch yarns utilized, and fabric structure, such as stitch size. The compressive force provided by such conventional fabrics and garments is static. That is, the amount of compressive force applied to a wearer of the garment cannot be changed. Thus, the compressive force applied by such conventional garments can be limited to the initial fabric structure and characteristics. In addition, the actual compressive force applied also depends on the fit of the garment on an anatomical area, for example, a leg, being compressed. As a result, an accurate application of a particular compressive force may be difficult to achieve using such conventional compression garments.

Such static compressive force garments can have other disadvantages. For example, to achieve different amounts of compressive force on different points, or zones, on a wearer using such garments, a different garment having a different defined compressive force capability may need to be donned by the wearer at each zone. Efforts to achieve a high compressive force value on a wearer may require a single layer high compression garment or multiple layers of lower compression garments. Since the compressive force of a static compressive force garment cannot change, in order to vary compressive force at a particular location or along an entire anatomical area (such as a limb), the single or multiple garments must be changed. Another disadvantage of conventional static compressive force garments is that the initial compressive force of such a garment can often diminish over time as a consequence of yarn fatigue. Yam fatigue can be defined as the weakening of a yarn caused by a loss of some of its ability to recover to its original shape or size after being deformed repeatedly.

Some conventional compression products utilize air pumps and bladders to provide compressive force to an anatomical area. For example, an air bladder can be strapped to a wearer's limb, and a desired amount of air pressure can be pumped into, or released from, the bladder. Air bladders may have the ability to apply compression in different zones, for example, in the foot, ankle, calf, and/or thigh of a wearer. Bladders may be regulated to provide a different amount of compression in various zones, for example, progressively more compression in the ankle, calf, and thigh of a wearer. Bladders may be adapted to provide constant pressure or intermittent pressure in one or more zones, as well as variable pressure with one or more zones. However, such air pump/bladder devices can have disadvantages. For example, such air bladder devices can be bulky to wear and may be uncomfortable due to prevention of heat dissipation from underneath plastic or vinyl materials comprising the bladders. In addition, an air pump/bladder may not be usable in some anatomical locations, such around the instep of a foot while a wearer is mobile.

Thus there is a need for a compression fabric and garment that can be dynamically and accurately adjusted while being worn.

### BRIEF DESCRIPTION OF THE DRAWINGS

FIG. 1 is a view of a compression adjustable garment having a compression element tube in selected areas of the garment and an air pump in an embodiment of the present invention.

FIG. 2 is a view of a compression adjustable garment having a compression element tube throughout the garment and an air pump in an embodiment of the present invention.

FIG. 3 is a view of a compression adjustable garment having an electrically stimulatable yarn in selected areas of the garment and an electrical stimulator in an embodiment of the present invention.

FIG. 4 is a view of a compression adjustable garment having an electrically stimulatable yarn throughout the garment and an electrical stimulator in an embodiment of the present invention.

FIG. 5 is a view of a compression adjustable garment having a compression element tube in selected areas of the garment, air bladders in those selected areas of the garment, and an air pump in an embodiment of the present invention.

FIG. 6 is a view of a rib stitch knit pattern useful for knitting a compression element in an embodiment of the present invention.

FIG. 7 is a view of a tuck stitch knit pattern useful for holding in an inlaid compression element in an embodiment of the present invention.

### SUMMARY

Some embodiments of the present invention can include a compression adjustable fabric and/or garment, system, and/or method. In some embodiments, the compression adjustable fabric can include a compression element (a) integrated into a fabric structure, (b) having a compressive pressure capability independent from compressive pressure capabilities inherent in the fabric structure, and (c) adjustable to provide various compressive pressures. The compression adjustable fabric may further comprise a compression adjustable garment. In such a garment, the compressive pressure provided by the garment can be adjusted in all or part of the garment while the garment is being worn. The compressive pressures in different parts of the garment can be independently adjustable.

In some embodiments, the compression element can further comprise an inflatable tube, and the inflation and deflation of the tube can be controlled by a pump. In certain embodiments, the inflatable tube can comprise an internal diameter of about 1 mm. In certain embodiments, the fabric can include a plurality of separate, unconnected inflatable tubes, and the amount of pressure in each of the tubes can be independently controlled by the pump. Alternatively, the amount of pressure in each of the tubes can be controlled by a different pump. In an embodiment comprising a compression adjustable garment, the pump can be miniaturized, attachable to the garment, and wearable with the garment.

In some embodiments, the compression element can further comprise an electrically stimulatable yarn, and the contraction and relaxation of the yarn can be controlled by an electrical stimulator. The electrically stimulatable yarn can comprise magnetic properties such that when the yarn is electrically stimulated, a length of the yarn is reduced, thereby increasing compressive pressure applied by the fabric. In certain embodiments, the fabric can include a plurality of separate, unconnected electrically stimulatable yarns, and the length of each of the yarns can be independently controlled by the electrical stimulator. Alternatively, the length of each of the yarns can be independently controlled by a different electrical stimulator. In an embodiment comprising a compression adjustable garment, the electrical stimulator can be miniaturized, attachable to the garment, and wearable with the garment.

In some embodiments of a compression adjustable fabric and/or garment, the compression element can further comprise a chemically stimulatable yarn. In some embodiments, the compression element can comprise an inflatable tube, an electrically stimulatable yarn, a chemically stimulatable yarn, or a combination thereof.

In some embodiments, the compression element can be integrated into the fabric structure by being knit into the fabric structure, such as in a rib knit construction. In some embodiments, the compression element can be integrated into the fabric structure by being laid in the fabric structure, such as in a tuck stitch construction. In particular embodiments, the compression element can be knit into a first portion of the fabric and laid in a second portion of the fabric. In some embodiments, the compression adjustable fabric and/or garment can further include a sensor adapted to monitor compressive pressure applied by the compression adjustable garment.

Some embodiments of the present invention can include a compression adjustable garment system. Such a system can comprise a compression adjustable garment comprising a compression element (a) integrated into a fabric structure of the garment, (b) having a compressive pressure capability independent from compressive pressure capabilities inherent in the fabric structure, and (c) adjustable to provide various compressive pressures. In embodiments of the system, the compression element can be integrated into the fabric structure by being knit into the fabric structure, by being laid in the fabric structure, or both.

Some embodiments of a compression adjustable garment system can further include a sensor adapted to detect changes in one or more health indicators, and a microprocessor adapted to receive and analyze health indicator data from the sensor and formulate a command for adjusting compressive pressure in the garment. The compression element can be adapted to receive the command and adjust the compressive pressure in the garment in response to the health indicator data. In one illustrative embodiment, the sensor can include a blood flow sensing system. The compression element can comprise an inflatable tube controllable by a pump, and adjustment of the compressive pressure in the garment can comprise adjustment by the pump of an amount of inflation, or deflation, of the inflatable tube in response to the level of blood flow detected. In another illustrative embodiment, the compression element can comprise an electrically stimulatable yarn controllable by an electrical stimulator, and adjustment of the compressive pressure in the garment can comprise adjustment by the electrical stimulator of an amount of longitudinal contraction of the electrically stimulatable yarn in response to the level of blood flow detected. Another embodiment of such a system can include a compressive pressure air bladder wearable over the compression adjustable garment, and the compressive pressure provided by the air bladder can be controlled by a pump separate from the garment.

Some embodiments of the present invention can include a method, comprising providing a compression adjustable garment comprising a compression element (a) integrated into portions of a fabric structure, (b) having a compressive pressure capability independent from compressive pressure capabilities inherent in the fabric structure, and (c) adjustable to provide various compressive pressures. The method can further include adjusting the compressive pressure provided in a first portion of the garment while the garment is being worn, and adjusting the compressive pressure provided in a second portion of the garment independently of the adjusting of the compressive pressure provided in a first portion of the garment.

In some embodiments of such a method, the compression element can be an inflatable tube, and independently adjusting the compressive pressures can include controlling inflation and deflation of the tube with a pump. In some embodiments of such a method, the compression element can be an electrically stimulatable yarn, and independently adjusting the compressive pressures can include controlling the contraction and relaxation of the yarn with an electrical stimulator. Some embodiments of a method can further include monitoring compressive pressure applied by the compression adjustable garment with a sensor. Some embodiments of a method can further include monitoring blood flow underneath the compression adjustable garment, and adjusting the compressive pressure provided by the garment in response to the level of blood flow detected.

Features of a compression adjustable fabric and/or garment, system, and/or method may be accomplished singularly, or in combination, in one or more of the embodiments of the present invention. As will be realized by those of skill in the art, many different embodiments of a compression adjustable fabric and/or garment, system, and/or method are possible. Additional uses, advantages, and features of aspects of the present invention are set forth in the illustrative embodiments discussed in the detailed description herein and will become more apparent to those skilled in the art upon examination of the following.

### DETAILED DESCRIPTION

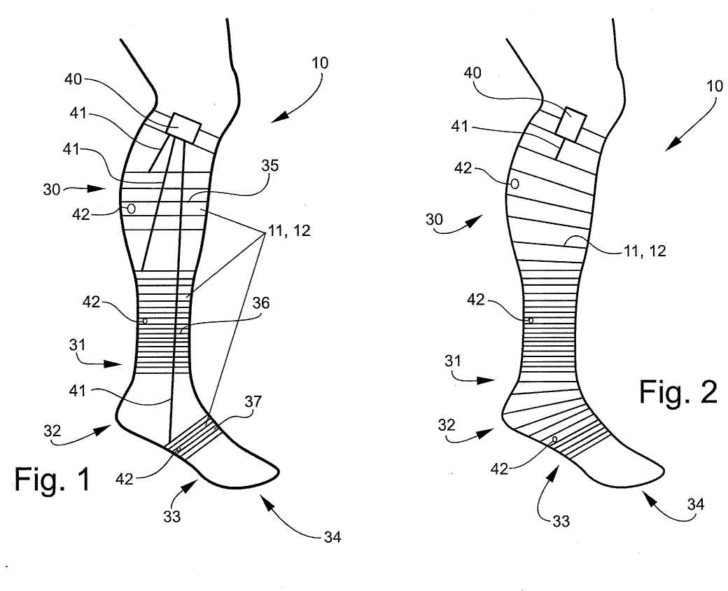

Some embodiments of the present invention can provide a compression adjustable fabric and/or garments made from compression adjustable fabric. FIGS. 1-7 illustrate embodiments of such compression adjustable fabric and garments. As shown in FIGS. 1-7, some embodiments of the compression adjustable fabric and garments 10 can include a compression element 11 integrated with the fabric structure and having variable compressive capabilities. For purposes herein, fabric structure is defined as the construction elements of a fabric including elements such as, but not limited to, yarn type, size, and performance characteristics, knit pattern, and stitch size. In some embodiments, the compression element 11 can have compressive capabilities independent from and/or in addition to the compressive capabilities of the fabric structure alone. In such a fabric, and in a garment made therefrom, compression can be adjusted in all or part of the fabric or garment 10 while it is being worn. Having the ability to adjust compression in the fabric or garment 10 while it is being worn can allow flexibility in treatment of certain patient conditions that can optimize treatment effectiveness and outcomes.

In some embodiments, the compression element 11 can comprise an inflatable tube 12 incorporated into the fabric structure, as shown in FIGS. 1 and 2. The inflatable tube 12 can have a small diameter, for example, an internal diameter of approximately 1 mm. In certain embodiments, the tube 12 can have a diameter smaller or larger than 1 mm. The diameter of the compression tube 12 can depend on various factors, including, for example, the material(s) used to make the tube 12, the desired compressive pressure capability in the tube 12; the intended use of the compression fabric 10 in which the tube 12 is incorporated; and/or the anatomical area to which the garment 10 is to be applied. The tube 12 can comprise various materials suitable for expanding and contracting the internal diameter of the tube 12 in response to varying amounts of inflation to provide varying compressive forces. Such materials may include polypropylene, polyurethane, or other plastics, polymers, and/or materials. In certain embodiments, the compression element tube 12 can comprise layers of laminated materials, which can facilitate expansion and contraction of the tube diameter. In certain embodiments, the compression element tube 12 can contain a fluid, gas, or other material adapted to enhance expansion and contraction performance of the tube 12. The material can be the same material or a different material as the primary material used for inflating the compression element tube 12. For example, in one embodiment, the primary inflation material can be air, and the expansion enhancing material can be another gas that enhances the expansion capability of air. The expansion enhancing material can be one that is adapted to remain in the compression tube 12 when the primary inflation material is removed so as to deflate the tube 12. Alternatively, the expansion enhancing material can be removed from the tube 12 along with the primary inflation material during deflation of the tube 12.

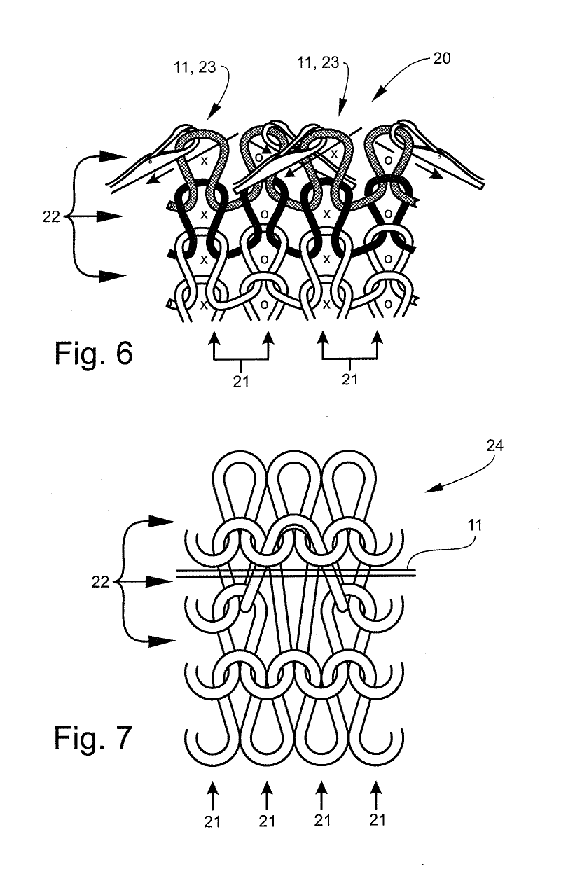

In some embodiments, the compression element tube 12 can be incorporated into the fabric structure by knitting the tube 12 into the fabric 10 as the fabric 10 is being knit. For example, the compression element tube 12 may be knit into the fabric 10 in a rib stitch construction 20, such as a 1×1 rib stitch pattern, utilizing a multi-feed circular knitting machine. FIG. 6 illustrates such a rib stitch knit pattern 20. A rib stitch 20 is defined as a knitting stitch characterized by alternation of wales 21 on the two sides of the fabric 10. Two rows of needles are employed, one knitting the wales 21 of the face, and the other knitting the back wales 21. (Fairchild's Dictionary of Textiles, 7^{th} Edition, p. 472). A knitting pattern can include wales 21 and courses 22. Wales are defined as a series of loops formed by the action of one needle in successive courses along a fabric length. (Fairchild's Dictionary of Textiles, 7^{th} Edition, p. 619). Courses are defined as rows of loops or stitches running across the width of a knitted fabric. (Fairchild's Dictionary of Textiles, 7^{th} Edition, p. 144). In such a rib stitch pattern 20, the compression element tube 12 can be knit so as to form the alternating wales 21 comprising one of the “rib” portions 23 of the fabric 10. In this way, the compression element tube 12 can be integrated with the fabric structure.

In other embodiments, the compression element tube 12 can be “laid in” in the fabric structure. In a “laid in” fabric, a base structure of knitted or overlapped (warp knitted) threads hold in position other non-knitted threads which are incorporated, or “laid in,” into the structure during the same knitting cycle. Although an inlaid yarn is not formed into a knitted loop, the base fabric structure can utilize various knitting stitches, for example, a tuck stitch 24, to hold the inlaid yarn in place. A tuck stitch 24 is defined as a knitting stitch that produces tuck or openwork effects by having certain needles hold more than one stitch at a time. (Fairchild's Dictionary of Textiles, 7^{th} Edition, p. 591). In a tuck stitch 24, the needles in the upper knitting position do not knit, but an extra loop of yarn is laid over the needles. The extra loop is not intermeshed through the old loop but is tucked in behind it on the reverse side of the stitch. When these needles are returned to a knitting position, all the loops on the needle are knit in a single stitch. (David J. Spencer, Knitting Technology, p. 59). As shown in FIG. 7, such a tick stitch knit pattern 24 can incorporate the compression element tube 12 between courses 22. In this manner, the tuck stitches 24 can help hold the tube 12 in place within the fabric structure.

In certain embodiments, the compression adjustable fabric 10 can include a combination of the compression tube element 11 knitted in to all or portions of the fabric 10 and laid in all or portions of the fabric 10. The portions of the fabric 10 into which the compression element tube 12 is integrally knit and into which the compression element tube 12 is laid in can be the same or different portions of the fabric 10.

In some embodiments, for example, as shown in FIG. 1, the compression element 11 can include a plurality of separate, unconnected tubes 12. For example, the calf portion 30 of the compression adjustable anti-embolism stocking 10 can include a first tube 35, the ankle portion 31 can include a second tube 36 separate from the first tube 35, and the foot portion 33 can include a third tube 37 separate from the first and second tubes 35, 36, respectively. Each of the first, second, and third tubes 35, 36, 37, respectively, can be independently connected to a pump 40 with a pump connecting tube 41. In this way, the amount of compressive pressure in each of the first, second, and third tubes 35, 36, 37, respectively, can be adjusted independently so as to separately vary the amount of compressive pressure at different locations in the garment 10. In particular embodiments having a plurality of independent tubes 12, the pressure in each tube 12 can be controlled by a separate pump 40.

In embodiments having a plurality of independent tubes 12, the pressure in each tube 12 can be controlled by the same pump 40. Such a pump 40 can be a miniaturized pump 40. Such a pump 40 can include a regulator mechanism for separately controlling flow of inflation material, such as air, into or out of different tubes 12. In still other embodiments, selected ones of the plurality of tubes 12 can be connected to each other. In certain embodiments, the first, second, and third tubes 35, 36, 37, respectively, can each be a continuous tube 12 within the respective separate regions of the garment 10. In other embodiments, the first tube 35 in the calf region 30 can comprise a plurality of independent tubes 12, and each tube 12 in the calf first tube region 30 can be independently connected to the pump 40, or to separate, miniaturized pumps 40. Likewise, the second tube 36 in the ankle region 31 and the third tube 37 in the foot region 33 can comprise a plurality of independent tubes 12, and each of those tubes 12 can be independently connected to the pump 40, or to separate, miniaturized pumps 40.

In other embodiments, the compression element tube 12 can be a single continuous tube 12 integrated into a continuous portion of the fabric 10, as shown in FIG. 2. In this way, a single change of pressure within the tube 12 can adjust the compression level of the continuous portion of the garment 10. In the embodiment shown in FIG. 2, the continuous portion of the garment 10 comprises the calf 30, ankle 31, heel 32, and foot 33 regions, but not the toe 34 region of the lower leg compressive pressure garment 10. In other embodiments, the continuous portion of the garment 10 can be the entire garment 10, for example, in this instance comprising the calf 30, heel 31, ankle 32, foot 33, and toe 34 regions.

In operation, inflation material such as air can be pumped into, or released from, the compression element tube 12 in order to provide varying amounts of pressure within the tube(s) 12 and thereby provide correspondingly varying degrees of compressive pressure and/or longitudinal stretch to the tube 12. For example, increasing the amount of pressure in the tube 12 can increase the diameter of the tube 12 and may directly increase the compressive pressure on a wearer's body adjacent the position where the tube 12 has increased pressure. In certain embodiments, increasing the amount of pressure in the tube 12 (and thus increasing the diameter of the tube 12) may also cause the tube 12 to decrease its ability to stretch, or elongate, along its longitudinal axis. As a result, the decreased stretch characteristics of the tube 12 may further increase the compressive pressure on a wearer's body.

In embodiments in which the compression element tube 12 is “laid in” the fabric structure, increasing the pressure within the tube 12 can increase the diameter of the tube 12 and decrease elongation, or longitudinal stretch, of the tube 12 so as to provide increased compressive pressure in the regions of the garment 10 comprising such a fabric 10. In embodiments in which the compression element tube 12 is knitted into the fabric structure, increasing the pressure within the tube 12 can increase the diameter of the tube 12 and decrease elongation, or longitudinal stretch, of the tube 12 so as to provide increased compressive pressure in the regions of the garment 10 comprising such a fabric 10.

Inflation material such as air can be pumped into the compression element tube 12 with various types and sizes of pumps 40. For example, the pump 40 may be separate from the compression adjustable garment 10 and attached to a port on the garment 10 when increasing the compressive pressure of the garment 10 is desired. Alternatively, the pump 40 can be miniaturized such that it can be attached to the garment 10 and worn unobtrusively by the wearer of the garment 10.

In certain embodiments, the compressive pressure garment 10 and/or the pump 40 can include a pressure monitoring capability. That is, the compressive pressure garment 10 and/or pump 40 may include a sensor 42 that monitors the pressure within the tube 12 and/or the compressive pressure being applied by the compression adjustable garment 10. Such a pressure monitoring capability can provide assurance to the garment wearer that the proper amount of pressure is being applied, and can be utilized to monitor a change in air pressure within the tube 12 and compressive pressure of the garment 10 over time and when being adjusted.

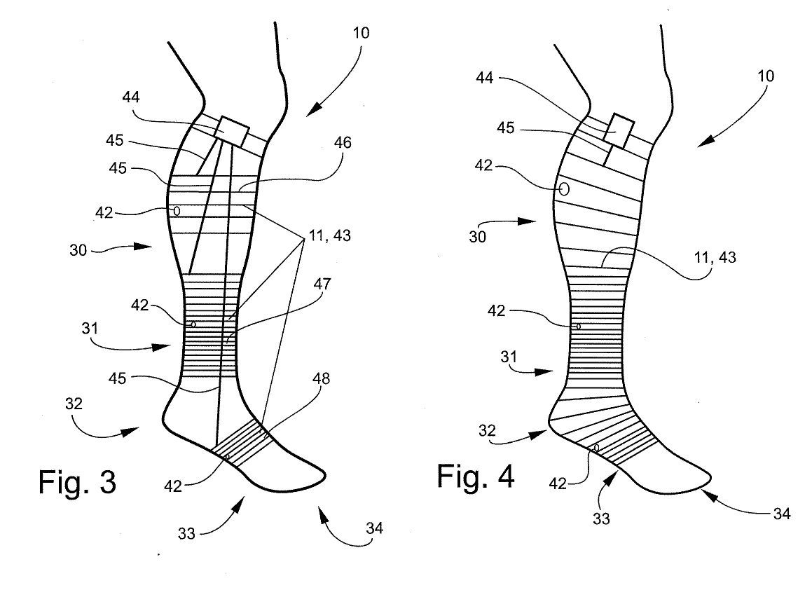

In an alternative embodiment, the compression element 11 can comprise an electrically stimulatable yarn 43, as shown in FIGS. 3 and 4. The electrically stimulatable yarn 43 can be “laid in” or knit into the fabric structure, as described herein for the compression element tube 12. An electrically stimulatable yarn 43 can comprise magnetic properties such that when the yarn 43 is electrically stimulated, it contracts longitudinally so as to reduce its length. In embodiments of the compression adjustable fabric 10 in which the electrically stimulatable yarn 43 is “laid in” or knit into the fabric structure, an electrically stimulated reduction in yarn length can cause an increase in the level of compression on the areas of a wearer underneath the region(s) in the garment 10 in which the electrically stimulatable yarn 43 is located.

In operation, varying levels of electrical stimulation can be provided to the electrically stimulatable yarn 43. As a result, an electrically stimulated yarn 43 can be shortened by varying amounts, and thereby provide correspondingly varying degrees of compressive pressure to an underlying anatomical structure of a person wearing the garment 10 having such an electrically stimulatable yarn 43 as the adjustable compression element 11.

In some embodiments, for example, as shown in FIG. 3, the compression element 11 can include a plurality of separate, electrically stimulatable yarns 43. For example, the calf portion 30 of the compression adjustable anti-embolism stocking 10 can include a first electrically stimulatable yarn 46, the ankle portion 31 can include a second electrically stimulatable yarn 47 separate from the first electrically stimulatable yarn 46, and the foot portion 33 can include a third electrically stimulatable yarn 48 separate from the first and second electrically stimulatable yarns 46, 47, respectively. Each of the first, second, and third electrically stimulatable yarns 46, 47, 48, respectively, can be independently connected to an electrical stimulator 44 with a stimulator connecting cable 45. In this way, the amount of compressive pressure in each of the first, second, and third electrically stimulatable yarns 46, 47, 48, respectively, can be adjusted independently so as to separately vary the amount of compressive pressure at different locations in the garment 10. In particular embodiments having a plurality of independent electrically stimulatable yarns 43, the pressure created by each electrically stimulatable yarn 43 can be controlled by a separate electric stimulator 44.

In embodiments having a plurality of independent electrically stimulatable yarns 43, the pressure in each electrically stimulatable yarn 43 can be controlled by the same electrical stimulator 44. Such an electrical stimulator 44 can be a miniaturized electrical stimulator 44. Such an electrical stimulator 44 can include a regulator mechanism for separately controlling electric current to different electrically stimulatable yarns 43. In still other embodiments, selected ones of the plurality of electrically stimulatable yarns 43 can be connected to each other. In certain embodiments, the first, second, and third electrically stimulatable yarns 46, 47, 48, respectively, can each be a continuous electrically stimulatable yarn 43 within the respective separate regions of the garment 10. In other embodiments, the first electrically stimulatable yarn 46 in the calf region 30 can comprise a plurality of independent electrically stimulatable yarns 43, and each electrically stimulatable yarn 43 in the calf region 30 can be independently connected to the electrical stimulator 44, or to separate, miniaturized electrical stimulators 44. Likewise, the second yarn 47 in the ankle region 31 and the third yarn 48 in the foot region 33 can comprise a plurality of independent electrically stimulatable yarns 43, and each of those electrically stimulatable yarns 43 can be independently connected to the electrical stimulator 44, or to separate, miniaturized electrical stimulators 44.

In other embodiments, the compression element 11 comprising the electrically stimulatable yarn 43 can be a single, continuous electrically stimulatable yarn 43 integrated into a continuous portion of the fabric structure, as shown in FIG. 4. In this way, a single change of pressure caused by the electrically stimulatable yarn 43 can adjust the compression level of the continuous portion of the garment 10. In the embodiment shown in FIG. 4, the continuous portion of the garment 10 comprises the calf 30, ankle 31, heel 32, and foot 33 regions, but not the toe 34 region of the lower leg compressive pressure garment 10. In other embodiments, the continuous portion of the garment 10 can be the entire garment 10, for example, in this instance comprising the calf 30, heel 31, ankle 32, foot 33, and toe 34 regions.

In some embodiments, the electrically stimulatable yarn 43 can be stimulated by an electrical stimulator 44, or generator, connected to the electrically stimulatable yarn 43 and worn with the compression adjustable garment 10. The electrical generator/stimulator 44 can include various energy sources, including, for example, a battery. The electrical generator/stimulator 44 can be separate from the compression adjustable garment 10 and attached to the electrically stimulatable yarn 43 when increasing the compressive pressure of the garment 10 is desired. Alternatively, the electrical generator 44 can be miniaturized such that it can be attached to the garment 10 and worn unobtrusively by the wearer of the garment 10.

In certain embodiments, the compressive pressure garment 10 and/or the electrical stimulator 44 can include monitoring capability. That is, the compressive pressure garment 10 and/or the electrical stimulator 44 may include the sensor 42 that monitors the compressive pressure being applied by the compression adjustable garment 10. Such a pressure monitoring capability can provide assurance to the garment wearer that the proper amount of pressure is being applied, and can be utilized to monitor a change in compressive pressure of the garment 10 over time and when being adjusted.

In another embodiment of the compression adjustable garment 10, the compression element 11 can comprise a yarn (not shown) that can be chemically stimulated to adjust compressive pressure of the yarn. The chemically stimulatable yarn can be “laid in” or knit into a fabric structure, as described herein related to the compression element tube 12 and the electrically stimulatable yarn 43. When chemically stimulated, the chemically stimulatable yarn can contract longitudinally so as to reduce its length, thereby causing an increase in compressive pressure in the portion(s) of the garment 10 in which the yarn is placed. In this way, compressive pressure in the garment 10 can be adjusted while being worn. The chemically stimulatable yarn can be a single, continuous yarn throughout the entire garment 10 or a portion of the garment 10. Alternatively, the garment 10 can include a plurality of independent chemically stimulatable yarns placed in desired portions of the garment 10.

In some embodiments of the present invention, the compressive pressure capabilities of the compression adjustable fabric and/or garment 10 can be provided by both the initial base fabric structure and the separate compression element 11. Compressive pressure capabilities of the initial base fabric structure can relate to various factors, including, for example, yarn type and size, characteristics of stretch yarns, such as spandex, utilized, and construction characteristics, such as stitch size and density. In addition to the static compressive pressure provided by the initial fabric structure, the separate compression element 11 can provide further compressive pressure capabilities, which can be adjustable.

In certain embodiments, the compression adjustable fabric and/or garment 10 can include the compression element tube 12, the electrically stimulatable yarn 43, and/or the chemically stimulatable yarn.

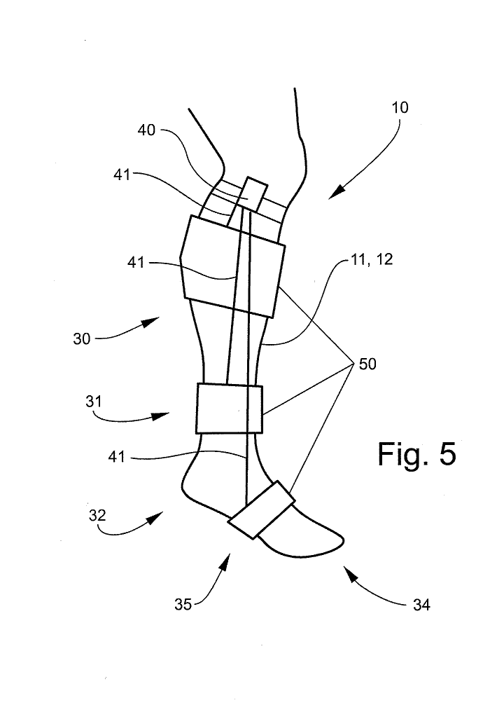

In particular embodiments, the compression adjustable fabric and/or garment 10 can include the combination of the separate compression element 11—such as the compression tube 12 and/or the electrically stimulatable yarn 43—with other mechanisms for increasing and controlling compressive pressure. For example, as shown in FIG. 5, one or more air bladders 50 can be constructed in, or overlaid on, the garment 10 comprising one or more of the compression element(s) described herein. Air can be pumped into the air bladder(s) 50 to change the pressure inside the air bladder(s) 50 and thus adjust the compressive pressure on the underlying anatomical structure. One or more of a plurality of air bladders 50 can be attached to the same pump 40 as the compression element tube 12, or to separate pumps 40. Accordingly, some embodiments of the present invention can include the garment 10 having inherent compressive capabilities; the compression element 11, such as the compression tube 12 and/or the electrically stimulatable yarn 43 integrated into the fabric structure, that can provide compressive capabilities in addition to those inherent to the fabric structure; and/or other compressive pressure mechanisms. At least one compression component of such a multi-component compressive fabric and/or garment can be adjustable.

Some embodiments of the compression adjustable fabric and garment 10 according to the present invention can provide advantages over conventional compression fabric and garments. For example, some embodiments of the present invention can provide the compression element 11 integrated with the fabric structure and having variable compressive capabilities. In certain embodiments, compression can be adjusted in all or in selected parts of the fabric or garment 10 in a dynamic fashion while it is being worn. Having the ability to adjust compression in the fabric or garment 10 while it is being worn can allow delivery of more accurate compressive pressures to a wearer than may be provided by fabrics or garments having static compressive pressures calibrated prior to being donned by the wearer. As a result, the compression fabric and garment 10 having dynamically adjustable compressive pressure capabilities can advantageously provide flexibility in treatment of certain patient conditions that can optimize treatment effectiveness and outcomes. Another advantage is that some embodiments of the present invention can provide the compression adjustable fabric and garment 10 that can be designed to fit any part of the body, thereby providing adjustable compression for virtually any anatomical area. For example, due to the integration of the compression element 11 in the fabric structure, particular embodiments of the compression adjustable garment 10 can be adapted to be worn about a person's foot. As a result, compressive pressure on the person's foot can be adjusted while the person is standing or ambulating. In this way, therapeutically optimal compressive pressures can be applied to the person's foot, and/or adjusted, to treat, for example, a venous stasis ulcer or other wound on the foot. In addition, such a wearable system can allow the garment 10 to be worn for extended periods while providing changes in compressive pressure only at intermittent, or infrequent, intervals.

Another advantage is that in some embodiments of the present invention the compressive pressure provided can be tailored to individual patients and for the same patient at different times. Another advantage is that in some embodiments of the present invention the compressive pressures applied can be varied within the same garment 10 for different anatomical areas covered by the same garment 10. Another advantage is that in some embodiments of the present invention the compression element(s) 11 can be incorporated into a variety of base fabrics having different yarn characteristics, thereby allowing for a broad range of fit and comfort options.

It is to be understood that an embodiment of the compression adjustable fabric 10 having the compression element 11 incorporated as described herein and the characteristics of such an embodiment of the fabric 10 are applicable to an embodiment of the compression adjustable garment 10 comprising that compression adjustable fabric 10.

Some embodiments of the compression adjustable fabric and/or garment 10 can be utilized in conjunction with a health monitoring and management system. Such a system is described in co-pending U.S. Patent Application entitled “Health Monitoring and Management System,” filed on Mar. 12, 2009, which application is incorporated herein by reference in its entirety. Some embodiments of such a health monitoring and management system can include the sensor 42 adapted to detect changes in one or more health indicators and transmit data related to the health indicators. In other embodiments, the health monitoring and management system can further include an interventional element adapted to receive a health intervention command and provide a health intervention related to the health indicators. In some embodiments, the system can further include a microprocessor adapted to receive and analyze the health indicator data transmitted by the sensor, formulate the health intervention command related to the health indicator data according to pre-determined parameters, and transmit the health intervention command to the interventional element. In certain embodiments, the pre-determined parameters can comprise a control algorithm configured to automatically control formulation of the health intervention command and transmission of the command to the interventional element. The health intervention command can be transmitted to the interventional element within a clinically relevant time period.

In some embodiments of the health monitoring and management system, the sensor and the microprocessor can be attachable to, or integrated with, a garment, such as an embodiment of the compression adjustable garment 10. In this way, the health intervention can comprise adjustment of the compressive pressure in the garment 10. As an example, such an embodiment of the compression adjustable garment 10 can include a blood flow sensing system and/or an edema sensing system. The interventional element can comprise the pump 40 connected to the compression element tube 12 or the electrical stimulator 44 connected to the electrically stimulatable yarn 43. The health intervention can comprise adjustment by the pump 40 of the amount of inflation material in the compression element tube 12, or adjustment by the electrical stimulator 44 of the amount of longitudinal contraction of the electrically stimulatable yarn 43, and thereby adjustment of the amount of compressive pressure applied by the compression adjustable garment 10, related to the level of edema and blood flow detected.

The present invention can include embodiments of a compression adjustable garment system. Such a system can comprise the compression adjustable garment 10 comprising the compression element 11 (a) integrated into a fabric structure of the garment 10, (b) having a compressive pressure capability independent from compressive pressure capabilities inherent in the fabric structure, and (c) adjustable to provide various compressive pressures. In embodiments of the compression adjustable garment system, the compression element 11 can be integrated into the fabric structure by being knit into the fabric structure, by being laid in the fabric structure, or both.

Some embodiments of a compression adjustable garment system can further include the sensor 42 adapted to detect changes in one or more health indicators, and a microprocessor adapted to receive and analyze health indicator data from the sensor 42 and formulate a command for adjusting compressive pressure in the garment 10. In certain embodiments, the microprocessor can be adapted to wirelessly receive the health indicator data from the sensor 42. The compression element 11 can be adapted to receive the command and adjust the compressive pressure in the garment 10 in response to the health indicator data. In one illustrative embodiment, the sensor 42 can include a blood flow sensing system. The compression element 11 can comprise the inflatable tube 12 controllable by the pump 40, and adjustment of the compressive pressure in the garment 10 can comprise adjustment by the pump 40 of an amount of inflation of the inflatable tube 12 in response to the level of blood flow detected. In another illustrative embodiment, the compression element 11 can comprise the electrically stimulatable yarn 43 controllable by the electrical stimulator 44, and adjustment of the compressive pressure in the garment 10 can comprise adjustment by the electrical stimulator 44 of an amount of longitudinal contraction of the electrically stimulatable yarn 43 in response to the level of blood flow detected. Another embodiment of such a system can include the compressive pressure air bladder 50 wearable over the compression adjustable garment 10, and the compressive pressure provided by the air bladder 50 can be controlled by the pump 40 separate from the garment 10.

The present invention can provide embodiments of a method of using the compression adjustable fabric and garments 10. Such methods of using the compression adjustable fabric and garments 10 can include combining various components of the compression adjustable fabric and/or garments 10 as described herein. For example, some embodiments of a method can include providing the compression adjustable garment 10 comprising the compression element 11 (a) integrated into portions of a fabric structure, (b) having a compressive pressure capability independent from compressive pressure capabilities inherent in the fabric structure, and (c) adjustable to provide various compressive pressures. The method can further include adjusting the compressive pressure provided in a first portion of the garment 10 while the garment is being worn, and adjusting the compressive pressure provided in a second portion of the garment 10 independently of the adjusting of the compressive pressure provided in a first portion of the garment 10.

In some embodiments of such a method, the compression element 11 can be then inflatable tube 12, and independently adjusting the compressive pressures can include controlling inflation and deflation of the tube 12 with the pump 40. In some embodiments of such a method, the compression element 11 can be the electrically stimulatable yarn 43, and independently adjusting the compressive pressures can include controlling the contraction and relaxation of the yarn 43 with the electrical stimulator 44. Some embodiments of a method can further include monitoring compressive pressure applied by the compression adjustable garment 10 with the sensor 42. Some embodiments of a method can further include monitoring blood flow underneath the compression adjustable garment 10, and adjusting the compressive pressure provided by the garment 10 in response to the level of blood flow detected.

Embodiments of the compression adjustable fabric and/or garment 10, system, and/or method can be utilized in a variety of applications. For example, some embodiments of the fabric, garment, system, and/or method can be utilized with humans, while others may be utilized for adjusting compressive pressure in animals. Some embodiments of the compression adjustable garment 10 can be utilized in care of wounds, either alone or in conjunction with other therapies. For example, the compression adjustable garment 10 can be adapted to adjust compressive pressure applied to a venous stasis ulcer or other wound in a person's foot while the garment 10 is being worn about the foot and the person is ambulating. In this way, optimal compressive pressures for enhancing blood flow and reducing edema can be applied and adjusted in a dynamic manner. In another application, certain embodiments of the compression adjustable garment 10 can be worn about a person's arm to adjust levels of compressive pressure about the arm to manage lymphedema. Embodiments of the compression adjustable garment 10, system, and/or method can be utilized to adjust levels of compressive pressure in virtually any anatomical region about which the garment 10 can be applied. Some embodiments may be particularly useful in managing various levels of compressive pressure in very small regions and/or in finely divided adjacent regions. Such use of this type of “micro-control” may be advantageous, for example, in controlling bleeding or drainage in and about a surgical site.

Features of the compression adjustable fabric and garments 10, a compression adjustable fabric system, and methods of using a compression adjustable fabric and garments 10 of the present invention may be accomplished singularly, or in combination, in one or more of the embodiments of the present invention. Although particular embodiments have been described, it should be recognized that these embodiments are merely illustrative of the principles of the present invention. Those of ordinary skill in the art will appreciate that a compression adjustable fabric and garments 10, a compression adjustable fabric system, and methods of using a compression adjustable fabric and garments of the present invention may be constructed and implemented in other ways and embodiments. Accordingly, the description herein should not be read as limiting the present invention, as other embodiments also fall within the scope of the present invention.

What is claimed is:

1. A compression adjustable fabric, comprising:
a compression element (a) integrated into a fabric structure, (b) having a compressive pressure capability independent from compressive pressure capabilities inherent in the fabric structure, and (c) adjustable to provide various compressive pressures.

2. The fabric of claim 1, wherein the compression adjustable fabric further comprises a compression adjustable garment, and
wherein the compressive pressure provided by the garment is adjustable in all or part of the garment while being worn.

3. The fabric of claim 2, wherein the compressive pressures in different parts of the garment are independently adjustable.

4. The fabric of claim 1, wherein the compression element further comprises an inflatable tube, the inflation and deflation of the tube controllable by a pump.

5. The fabric of claim 4, wherein the inflatable tube comprises an internal diameter of about 1 mm.

6. The fabric of claim 4, wherein the inflatable tube further comprises a plurality of separate, unconnected inflatable tubes, and
wherein an amount of pressure in each of the tubes is independently controllable by the pump.

7. The fabric of claim 6, wherein an amount of pressure in each of the tubes is controllable by a different pump.

8. The fabric of claim 4, wherein the compression adjustable fabric further comprises a compression adjustable garment, and
wherein the pump further comprises a miniaturized pump attachable to the garment and wearable with the garment.

9. The fabric of claim 1, wherein the compression element further comprises an electrically stimulatable yarn, the contraction and relaxation of the yarn controllable by an electrical stimulator.

10. The fabric of claim 9, wherein the electrically stimulatable yarn comprises magnetic properties such that when the yarn is electrically stimulated, a length of the yarn is reduced, thereby increasing compressive pressure applied by the fabric.

11. The fabric of claim 9, wherein the electrically stimulatable yarn further comprises a plurality of separate, unconnected electrically stimulatable yarns, and
wherein a length of each of the yarns is independently controllable by the electrical stimulator.

12. The fabric of claim 11, wherein a length of each of the yarns is independently controllable by a different electrical stimulator.

13. The fabric of claim 9, wherein the electrical stimulator further comprises a miniaturized electrical stimulator attachable to the garment and wearable with the garment.

14. The fabric of claim 1, wherein the compression element further comprises a chemically stimulatable yarn.

15. The fabric of claim 1, wherein the compression element further comprises an inflatable tube, an electrically stimulatable yarn, a chemically stimulatable yarn, or a combination thereof.

16. The fabric of claim 1, wherein the integration of the compression element into the fabric structure comprises the compression element knit into the fabric structure.

17. The fabric of claim 16, wherein the compression element is knit into the fabric structure in a rib knit construction.

18. The fabric of claim 1, wherein the integration of the compression element into the fabric structure comprises the compression element laid in the fabric structure.

19. The fabric of claim 18, wherein the compression element is laid in the fabric structure in a tuck stitch construction.

20. The fabric of claim 1, wherein the compression element is knit into a first portion of the fabric and laid in a second portion of the fabric.

21. The fabric of claim 2, further comprising a sensor adapted to monitor compressive pressure applied by the compression adjustable garment.

22. A compression adjustable garment system, comprising:
a compression adjustable garment comprising a compression element (a) integrated into a fabric structure of the garment, (b) having a compressive pressure capability independent from compressive pressure capabilities inherent in the fabric structure, and (c) adjustable to provide various compressive pressures.

23. The system of claim 22, further comprising:
a sensor adapted to detect changes in one or more health indicators;
a microprocessor adapted to receive and analyze health indicator data from the sensor and formulate a command for adjusting compressive pressure in the garment; and
the compression element adapted to receive the command and adjust the compressive pressure in the garment in response to the health indicator data.

24. The system of claim 23,
wherein the sensor further comprises a blood flow sensing system,
wherein the compression element further comprises an inflatable tube controllable by a pump, and
wherein adjustment of the compressive pressure in the garment comprises adjustment by the pump of an amount of inflation or deflation of the inflatable tube in response to the level of blood flow detected.

25. The system of claim 23,
wherein the sensor further comprises a blood flow sensing system,
wherein the compression element further comprises an electrically stimulatable yarn controllable by an electrical stimulator, and
wherein adjustment of the compressive pressure in the garment comprises adjustment by the electrical stimulator of an amount of longitudinal contraction of the electrically stimulatable yarn in response to the level of blood flow detected.

26. The system of claim 22, further comprising:
a compressive pressure air bladder wearable over the compression adjustable garment, the compressive pressure provided by the air bladder controllable by a pump separate from the garment.

27. The system of claim 22, wherein the integration of the compression element into the fabric structure comprises the compression element knit into the fabric structure.

28. The system of claim 22, wherein the integration of the compression element into the fabric structure comprises the compression element laid in the fabric structure.

29. A method, comprising:
providing a compression adjustable garment comprising a compression element (a) integrated into portions of a fabric structure, (b) having a compressive pressure capability independent from compressive pressure capabilities inherent in the fabric structure, and (c) adjustable to provide various compressive pressures;
adjusting the compressive pressure provided in a first portion of the garment while the garment is being worn; and
adjusting the compressive pressure provided in a second portion of the garment independently of the adjusting of the compressive pressure provided in a first portion of the garment.

30. The method of claim 29, the compression element further comprising an inflatable tube, wherein independently adjusting the compressive pressures further comprises controlling inflation and deflation of the tube with a pump.

31. The method of claim 29, the compression element further comprising an electrically stimulatable yarn, wherein independently adjusting the compressive pressures further comprises controlling the contraction and relaxation of the yarn with an electrical stimulator.

32. The method of claim 29, further comprising monitoring compressive pressure applied by the compression adjustable garment with a sensor.

33. The method of claim 29, further comprising:
monitoring blood flow underneath the compression adjustable garment; and
adjusting the compressive pressure provided by the garment in response to the level of blood flow detected.
